(19) United States
(12) Patent Application Publication
(10) Pub. No.: US 2018/0360511 A1
(43) Pub. Date: Dec. 20, 2018

(54) HUMAN BONE TREATMENT UNIT

(71) Applicant: INJE UNIVERSITY INDUSTRY-ACADEMIC COOPERATION FOUNDATION, Gimhae-si, Gyeongsangnam-do (KR)

(72) Inventors: Youn Uk KWON, Busan (KR); Gyu Min KONG, Busan (KR)

(21) Appl. No.: 16/061,376

(22) PCT Filed: Nov. 24, 2016
(86) PCT No.: PCT/KR2016/013591

§ 371 Date: Jun. 12, 2018

(30) Foreign Application Priority Data
Jul. 1, 2016 (KR) 10-2016-0083581

(51) Int. Cl. A61B 17/86

(52) U.S. Cl. CPC A61B 17/8615; A61B 17/8888; A61B 17/8625; A61B 17/862

(57) ABSTRACT

Provided is a human bone treatment unit. The human bone treatment unit is used to treat human bones, and includes: a main body part that passes through a human bone and a treatment rod and is then fastened to the bone; and a head part for enabling coupling between the bone and the treatment rod by means of an external force applied from the outside, wherein the head part is integrally formed with the main body part, provided with an engagement recess having a non-circular cross-section which is engaged with a screwdriver for creating the external force, and has an engagement surface having a non-circular cross-section that makes it possible to be additionally engaged with the screwdriver in a state in which the engagement between the screwdriver and the engagement recess is not possible.

### TECHNICAL FIELD

The present disclosure relates to a bone treatment unit used to treat human bones, and more particularly, a human bone treatment having an improved structure for facilitating the fastening and separation of a screw configured to couple a bone to be treated with a treatment rod, thereby enhancing treatment stability.

### BACKGROUND ART

In general, in order to treat a fractured bone, as shown in FIG. 1, a process of placing a treatment rod, such as a bone plate 101 or an intramedullary (IM) rod 102, at a fracture site and coupling the treatment rod and the bone with each other using a screw S has been performed.

However, according to this treatment method, when the screw S is fastened to the bone for a large amount of time, a coupling force between the screw S and the bone becomes large, so the screw S is not easily separated from the bone. Since the coupling force becomes larger than a force applied to the driver to rotate the screw S, a head of the screw S is damaged or broken. As a result, the screw S cannot be smoothly separated from the bone.

Because of this problem, the screw S may be separated from the bone while inflicting artificial damage on the bone to which the screw S is fastened, or the screw S cannot be separated from the bone at a desired time, thus frequently hindering patients' treatment stability.

### DESCRIPTION OF EMBODIMENTS

### Technical Problem

Accordingly, the present disclosure has been devised to solve the above-described problems. It is an object of the present disclosure to provide a human bone treatment unit capable of smoothly performing the fastening and separation of a screw that is fastened to a bone for treatment.

It is another object of the present disclosure to provide a human bone treatment unit capable of additionally attempting to separate a screw from a bone when a screw separation operation becomes impossible due to unexpected damage or breakage of the screw.

### Solution to Problem

According to an aspect of the present disclosure, a human bone treatment unit, which is used to treat human bones, includes: a main body part configured to pass through a human bone and a treatment rod and then fastened to the bone; and a head part configured to enable coupling between the bone and the treatment rod by means of an external force applied from the outside, wherein the head part is integrally formed with the main body part, includes an engagement recess having a non-circular cross-section that is engaged with a driver configured to create the external force, and has an engagement surface having a non-circular cross-section that makes it possible to be additionally engaged with the driver in a state in which the engagement between the driver and the engagement recess is not possible.

An inner circumference surface of the head part, which defines the engagement recess, may include a first inner surface in which the engagement recess is formed such that the driver is inserted into the first inner surface; and a second inner surface having a tapered shape to induce insertion of a guide thread that guides connection of the driver into the first inner surface and fastening between the bone and the main body part.

The inner circumferential surface of the head part may include a third inner surface prepared between the first inner surface and an interface surface connecting an interface between the head part and the main body part, the third inner surface having a tapered shape to guide introduction of the guide thread into the main body part.

The driver may include a first wrench part formed to be long in a same direction as an axial direction of the main body part, the first wrench part being engaged with the engagement recess; and a second wrench part formed around the first wrench part, the second wrench part having a receiving groove configured to locate the head part between the second wrench part and the first wrench part, the second wrench part being engaged with the engagement surface.

The first wrench part may be installed to be capable of moving relatively to the second wrench part. The human bone treatment unit may further include an elastic member located between the first wrench part and the second wrench part such that the first wrench part is elastically supported by the second wrench part.

### Advantageous Effects of Disclosure

A human bone treatment according to the present disclosure, which has the above-described configuration as described above, includes a head part having an engagement surface provided separately from an engagement recess, and can additionally perform a screw separation operation in preparation for unexpected damage or breakage of the engagement recess. Furthermore, the human bone treatment can doubly couple the engagement recess and the engagement surface with a driver to further increase a coupling force between the screw and the driver. Thus, an advantage of enabling smooth screw separation from a bone can be expected. This advantage enables the screw separation at an appropriate time. Accordingly, goals of ultimate medical services, which can obtain treatment stability and increase reliability of treatment, can be achieved.

### BRIEF DESCRIPTION OF DRAWINGS

FIG. 1 is a diagram for explaining problems of a bone treatment unit according to the related art.

FIG. 2 is a perspective view of a human bone treatment unit according to an embodiment of the present disclosure.

FIG. 3 is an exploded perspective view for explaining operating principles of an embodiment of the present disclosure.

FIG. 4 is a perspective view of a human bone treatment unit according to another embodiment of the present disclosure.

FIG. 5 is a perspective view of a human bone treatment unit according to another embodiment of the present disclosure.

FIG. 6 is an exploded perspective view of a human bone treatment unit according to another embodiment of the present disclosure.

FIG. 7 is a partial cross-sectional view of another embodiment of the present disclosure.

FIG. 8 is an exploded perspective view of a human bone treatment unit according to another embodiment of the present disclosure.

FIG. 9 is a partial cross-sectional view of another embodiment of the present disclosure.

FIG. 10 is a partial cross-sectional view showing an operation state according to another embodiment of the present disclosure.

### BEST MODE

Hereinafter, a human bone treatment unit according to an embodiment of the present disclosure will be described in detail with reference to the accompanying drawings.

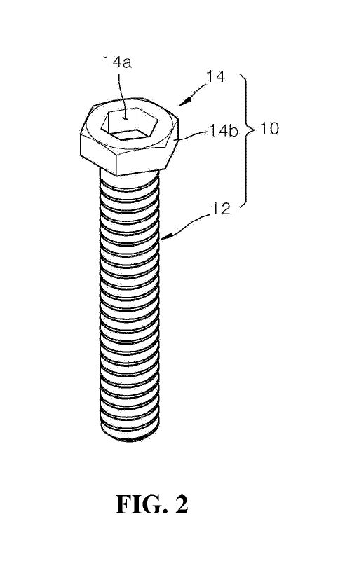

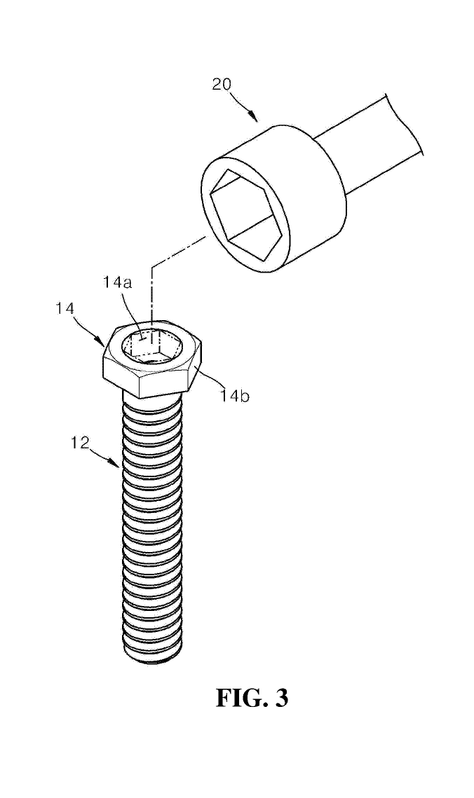

FIG. 2 is a perspective view of a human bone treatment unit according to an embodiment of the present disclosure. FIG. 3 is an exploded perspective view for explaining operating principles of an embodiment of the present disclosure.

As shown in FIGS. 2 and 3, the human bone treatment unit according to the present disclosure is used to treat fractured bones of a human and includes a main body part 12 and a head part 14.

A configuration including the main body part 12 and the head part 14 forms a structure of a screw 10. The human bone treatment unit according to the present disclosure may be implemented as an embodiment in which the human bone treatment unit includes only the screw 10 and an embodiment in which the human bone treatment unit includes the screw 10 and a driver 20 used together with the screw 10.

Hereinafter, the screw 10, which is a human bone treatment unit according to one of embodiments of the present disclosure, will be firstly described.

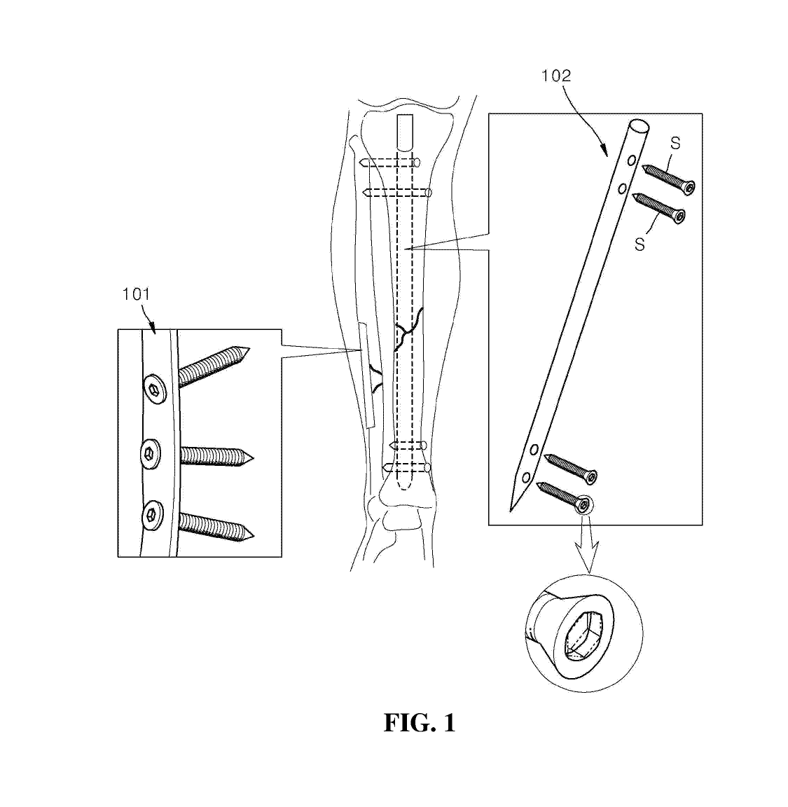

The main body part 12 included in the present embodiment is a part that passes through a human body and a treatment rod and is fastened to the bone. Here, the treatment rod refers to a bone plate (refer to 101 in FIG. 1) or an intramedullary (IM) rod (refer to 102 in FIG. 1), which is used together with the screw 10 for the treatment of a fractured site.

As shown in FIGS. 2 and 3, the head part 14 included in the present embodiment includes an engagement recess 14a having a non-circular cross-section and an engagement surface 14b having a non-circular cross-section, which is provided separately from the engagement recess 14a.

Here, since it is sufficient for the engagement recess 14a and the engagement surface 14b to have a non-circular cross-sectional shape, the engagement recess 14a and the engagement surface 14b are not limited to the illustrated hexagonal structure but may be implemented, for example, in a square or octagonal structure. Also, the engagement recess 14a may be implemented in a cross-shaped groove structure into which a cross-shaped protrusion of a cross-driver may be inserted.

Although not shown, the engagement recess 14a is a part into which a wrench part of a typical screw release mechanism (e.g., a protruding wrench) is inserted and to which a force is transmitted through the wrench part. Here, the wrench part includes the same outer surface shape as the engagement recess 14a.

As shown in FIG. 3, the engagement surface 14b enables the transfer of a force through an additional driver (20; a socket type wrench) that is provided separately from the typical screw release mechanism. In a normal state shown by a two-point chain line of FIG. 3, when the engagement recess 14a is damaged or broken as indicated by a solid line, the engagement surface 14b may additionally enable the separation of the screw 10 from the bone.

That is, when the screw 10 according to one embodiment of the present disclosure is fastened to the bone for treatment in a fixed state for a large amount of time, cases in which a coupling force between the bone and the screw 10 becomes larger than a force applied to the driver to rotate the screw 10 occur. Thus, the engagement recess 14a of the head part 14 is damaged or broken. As a result, the screw 10 cannot be smoothly separated from the bone.

However, according to an embodiment of the present disclosure, since the screw 10 includes the head part 14 having the engagement surface 14b provided separately from the engagement recess 14a, an operation of separating the screw 10 may be additionally performed in preparation for unexpected damage or breakage of the engagement recess 14a. Furthermore, by double coupling the engagement recess 14a and the engagement surface 14b with the driver 20, a coupling force between the screw 10 and the driver 20 may be further increased, thereby enabling smooth separation of the screw 10 from the bone. These advantages enable the separation of the screw 10 from the bone at an appropriate time point. Accordingly, goals of ultimate medical services, which can obtain treatment stability and increase reliability of treatment, can be achieved.

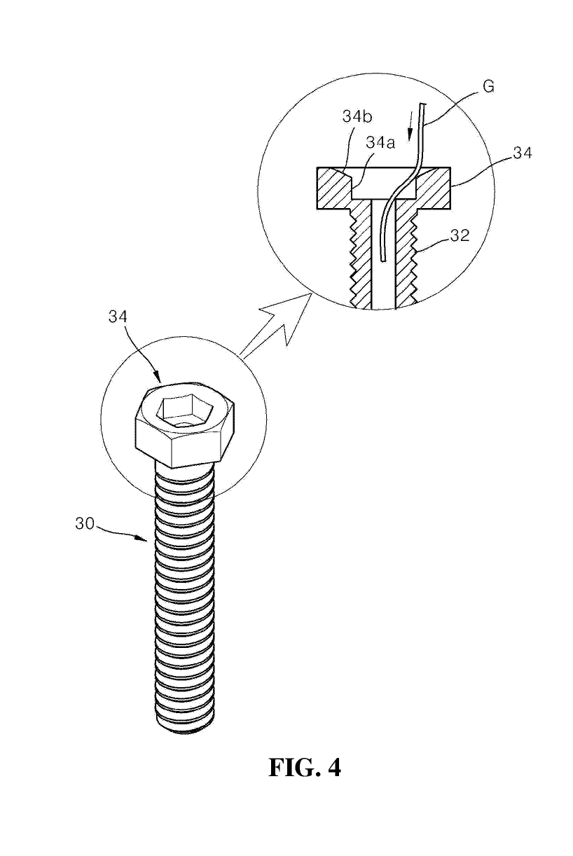

FIG. 4 is a perspective view of a human bone treatment unit according to another embodiment of the present disclosure.

In addition to the structure according to the above-described embodiment, in the embodiment shown in FIG. 4, an inner circumferential surface of a head part 34 forming an engagement recess includes a first inner surface 34a and a second inner surface 34b.

That is, as shown in an enlarged portion of FIG. 4, the first inner surface 34a is a portion in which the engagement recess is formed so that a driver may be inserted into the first inner surface 34a. The second inner surface 34b is a portion having a tapered shape so that a guide thread G may be guided into a main body part 32.

Here, the guide thread G serves to guide the connection of the driver to a side of the first inner surface 34a and fastening between the bone and the main body part 32.

In the present embodiment having the above-described configuration, the guide thread G for guiding the driver connected to the engagement recess or a position of the fastening of a screw to the bone may be smoothly guided by the second inner surface 34b having the tapered shape into a through hole of the main body part 32. Thus, an operation of fastening the screw to the bone and separating the screw from the bone may be smoothly performed.

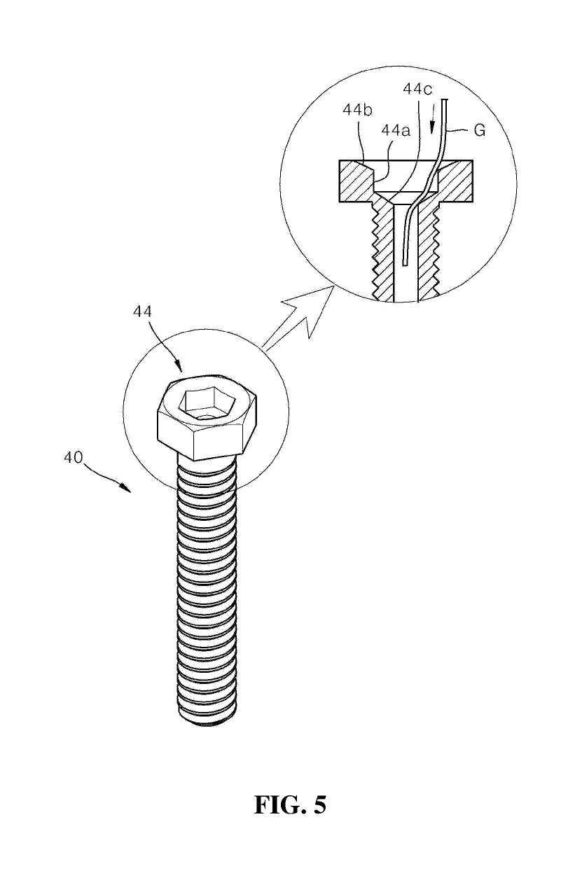

FIG. 5 is a perspective view of a human bone treatment unit according to another embodiment of the present disclosure.

In addition to the structure of the embodiment shown in FIG. 4, the embodiment shown in FIG. 5 has a head part structure that further includes a third inner surface 44c.

That is, an inner surface of a head part 44 adopted in the present embodiment has a first inner surface 44a and a second inner surface 44b, which are adopted in the above-described embodiment, and further has the third inner surface 44c, which is prepared in a tapered shape between the first inner surface 44a and an interface surface. Here, the interface surface refers to a portion connecting an interface between the head part 44 and a main body part.

In the present embodiment having the above-described configuration, as shown in an exploded cross-sectional view of FIG. 5, a guide thread may be guided more smoothly into a through hole of the main body part.

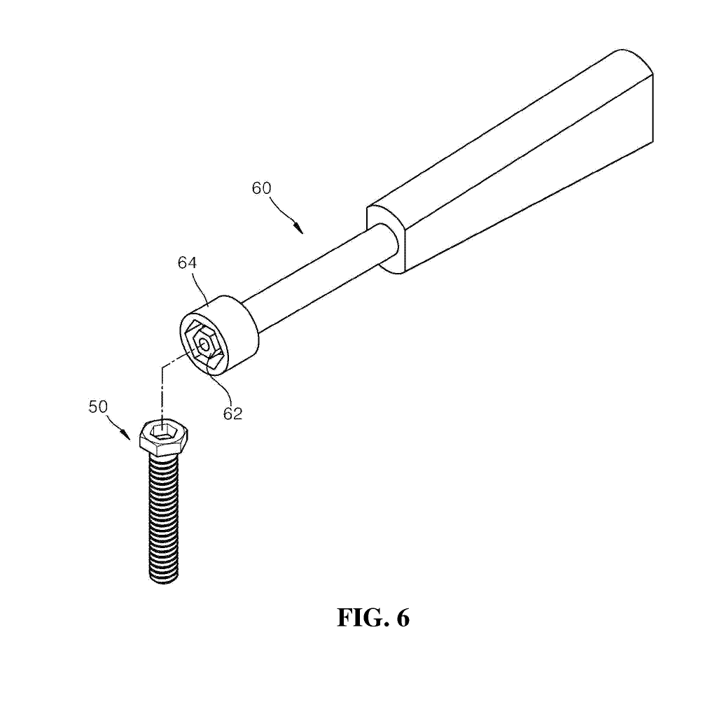

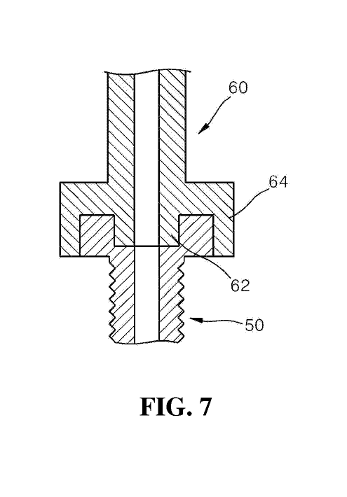

FIG. 6 is an exploded perspective view of a human bone treatment unit according to another embodiment of the present disclosure. FIG. 7 is a partial cross-sectional view of another embodiment of the present disclosure.

The embodiment shown in FIG. 6 pertains to an embodiment in which the human bone treatment unit according to the present disclosure includes a screw 50 and a driver 60.

Since the screw 50 has fully been described above, the driver 60 will be described in detail below.

The driver 60 adopted in the present embodiment includes a first wrench part 62 and a second wrench part 64.

The first wrench part 62 is a portion that is formed to be long in the same direction as an axial direction of the main body part and engaged with the engagement recess. The second wrench part 64 is formed around the first wrench part so that a head part of the screw may be located between the second wrench part 64 and the first wrench part. Here, the second wrench part may have an inner circumferential surface structure having a non-circular cross-section so that the second wrench part 64 may be engaged with an engagement surface of the head part.

In the present embodiment having the above-described configuration, the screw 50 may be separated by using the driver 60 including the first wrench part 62 and the second wrench part 64. Thus, a release force greater than a coupling force between the screw 50 and a bone may be applied to the screw. As a result, the screw 50 may be separated at an appropriate treatment time point, thereby further maximizing treatment stability.

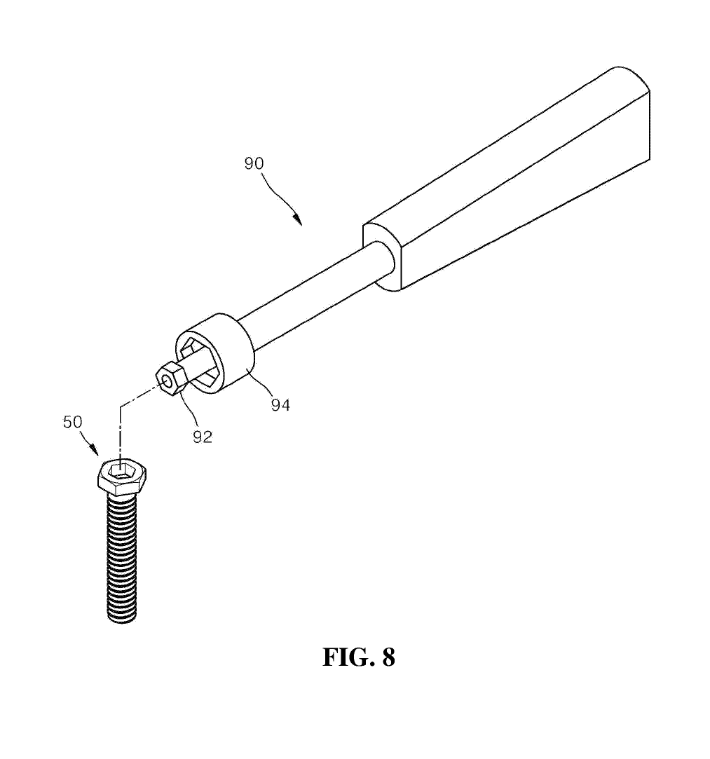

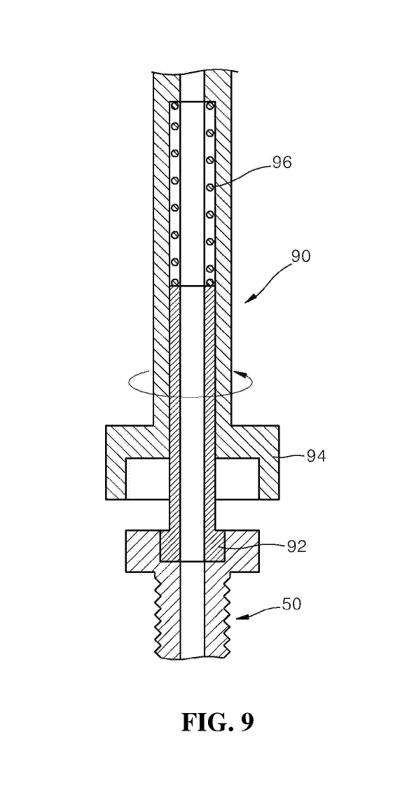

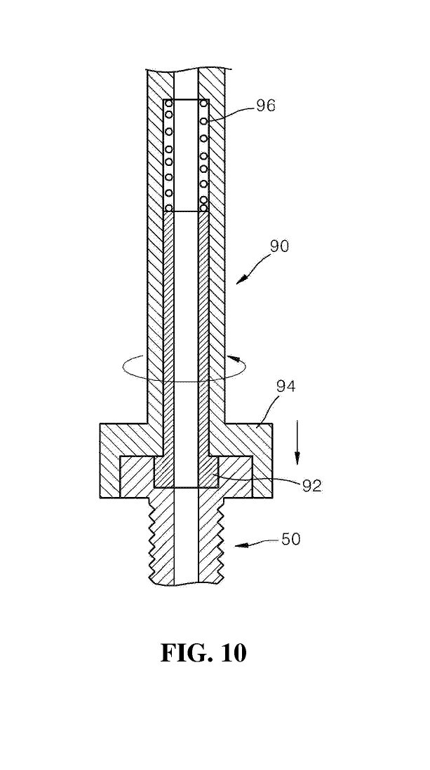

FIG. 8 is an exploded perspective view of a human bone treatment unit according to another embodiment of the present disclosure. FIG. 9 is a partial cross-sectional view of another embodiment of the present disclosure. FIG. 10 is a partial cross-sectional showing an operation state of another embodiment of the present disclosure.

As shown in FIGS. 8 to 10, unlike in the embodiment shown in FIGS. 6 and 7, the human bone treatment unit according to the present embodiment is configured such that a first wrench part 92 may move relatively to a second wrench part 94.

That is, the first wrench part 92 may be located in a position protruding from the second wrench part 94 as shown in FIGS. 8 and 9 or located in a retreated position toward the second wrench part 94 as shown in FIG. 10.

For example, when the separation of the screw 80 from a bone is enabled only by the first wrench part 92, as shown in FIGS. 8 and 9, the first wrench part 92 is engaged with an engagement recess of the screw 80 and used without engagement between the second wrench part 94 and the screw 80. When the screw 80 is not smoothly separated only by the first wrench part 920 during the use, as shown in FIG. 10, as shown in FIG. 10, the second wrench part 94 may be applied with pressure toward the first wrench part 92 so that a head part of the screw 80 may be located between the first wrench part 92 and the second wrench part 94.

In the present embodiment having the above-described configuration, the first wrench part 92 is installed to be capable of moving relatively to the second wrench part 94, and elastically supported by the second wrench part 94 using an elastic member 96. In this state, the first wrench part 92 may be used alone or used together with the second wrench part 94 as needed. Accordingly, user convenience may be enhanced, and treatment stability may be maximized.

While the present disclosure has been particularly shown and described with reference to exemplary embodiments thereof, the present disclosure is not limited to the above-described embodiments, and it will be understood by those of ordinary skill in the art that various changes and modifications may be made therein without departing from the spirit and scope of the present disclosure as defined by the following claims.

1. A human bone treatment unit which is used to treat human bones, the human bone treatment unit comprising:
a main body part configured to pass through a human bone and a treatment rod and to be fastened to the bone; and
a head part configured to enable coupling between the bone and the treatment rod by an external force applied from outside, wherein the head part is integrally formed with the main body part, includes an engagement recess having a non-circular cross-section that is engaged with a driver configured to create the external force, and has an engagement surface having a non-circular cross-section that enables additional engagement with the driver when the engagement between the driver and the engagement recess is not possible.

2. The human bone treatment unit of claim 1, wherein an inner circumferential surface of the head part, which defines the engagement recess, comprises:
a first inner surface in which the engagement recess is formed such that the driver is inserted into the first inner surface; and
a second inner surface having a tapered shape to induce insertion of a guide thread that guides connection of the driver into the first inner surface and fastening between the bone and the main body part.

3. The human bone treatment unit of claim 2, wherein the inner circumferential surface of the head part comprises a third inner surface prepared between the first inner surface and an interface surface connecting an interface between the head part and the main body part, the third inner surface having a tapered shape to guide introduction of the guide thread into the main body part.

4. The human bone treatment unit of claim 1, wherein the driver comprises:
a first wrench part formed to be long in a same direction as an axial direction of the main body part, the first wrench part being engaged with the engagement recess; and
a second wrench part formed around the first wrench part, the second wrench part having a receiving groove configured to locate the head part between the second wrench part and the first wrench part, the second wrench part being engaged with the engagement surface.

5. The human bone treatment unit of claim 4, wherein the first wrench part is provided to be capable of moving relatively to the second wrench part,
the human bone treatment unit further comprising an elastic member located between the first wrench part and the second wrench part such that the first wrench part is elastically supported by the second wrench part.
